(19) United States
(12) Patent Application Publication
(10) Pub. No.: US 2014/0213502 A1
(43) Pub. Date: Jul. 31, 2014

(54) CHEMICAL MODIFICATION OF APOLIPOPROTEIN MIMETIC PEPTIDES FOR THE PRODUCTION OF THERAPEUTIC AGENTS

(71) Applicants: Alan T. Remaley, Bethesda, MD (US); Marcelo Amar, Gaithersburg, MD (US); Denis Sviridov, Rockville, MD (US); Imoh Z. Ikpot, Rocherster, NY (US); David Osei-Hwedieh, Princeton, NY (US); Scott Turner, Oakland, CA (US)

(72) Inventors: Alan T. Remaley, Bethesda, MD (US); Marcelo Amar, Gaithersburg, MD (US); Denis Sviridov, Rockville, MD (US); Imoh Z. Ikpot, Rocherster, NY (US); David Osei-Hwedieh, Princeton, NY (US); Scott Turner, Oakland, CA (US)

(73) Assignees: United States Department of Health and Human Services, Bethesda, MD (US); KineMed, Inc., Emeryville, CA (US)

(21) Appl. No.: 14/114,386

(22) PCT Filed: Apr. 30, 2012
(86) PCT No.: PCT/US2012/035870

§ 371 Date: Feb. 18, 2014

Related U.S. Application Data
(60) Provisional application No. 61/480,986, filed on Apr. 29, 2011.

(51) Int. Cl. C07K 14/775

(52) U.S. Cl. CPC C07K 14/775
USPC 514/1.1; 530/300

(57) ABSTRACT

Hydrocarbon stapling of apolipoprotein mimetic peptides increases the helicity of the peptides, enhances their ability to promote cholesterol efflux by multiple mechanisms and makes them resistant to proteolysis. Hydrocarbon stapled amphipathic helical peptides are useful in the treatment of cardiovascular diseases and other disorders.

### CROSS REFERENCE TO RELATED APPLICATION

This application claims the benefit of U.S. Provisional Application No. 61/480,986, filed Apr. 29, 2011, which is incorporated herein by reference in its entirety.

### BACKGROUND

Apolipoprotein mimetic peptides are being investigated as possible therapeutic agents for the treatment of cardiovascular disease (Remaley, Thomas et al. 2003; Osei-Hwedieh, Amar et al. 2011), as well as other disorders associated with inflammation (Osei-Hwedieh, Amar et al. 2011; Yao, Oai et al. 2011). See also U.S. Patent Application Publication Nos. 2009/0276331 and 2010/0203099, both incorporated by reference in their entirety. These peptides reduce atherosclerosis in animal models and appear to be safe in early stage clinical trials (Wool, Reardon et al. 2008; Navab, Reddy et al. 2011; Watson, Weissbach et al. 2011).

Apolipoprotein mimetic peptides have similar biological properties as full length apolipoproteins, such as apoA-I, the main protein in High Density Lipoprotein (HDL). Intravenous infusion, once a week for 4-5 weeks, of recombinant or purified apoA-I reconstituted with phospholipids has been shown to reduce plaque volume in patients with acute coronary syndrome to a degree similar to that observed after several years of statin treatment (Nissen 2005; Tardif, Gregoire et al. 2007). A major limitation of the use of apoA-I is the cost to produce the large quantities needed for this type of treatment and hence the interest in the use of short synthetic mimetic peptides, which are potentially more economical to produce (Osei-Hwedieh, Amar et al. 2011). Another potential advantage of apolipoprotein mimetic peptides is that, when they are synthesized with D-amino acids, such as the D4F peptide, they are resistant to proteolysis and can reduce atherosclerosis in animal models when given orally (Buga, Frank et al. 2006; Bloedon, Dunbar et al. 2008). Clinical development of the D4F peptide, however, has been halted because of the potential for long-term tissue accumulation (Watson, Weissbach et al. 2011).

ApoA-1 and apolipoprotein mimetic peptides potentially have several different beneficial effects in preventing or reducing atherosclerosis (Osei-Hwedieh, Amar et al. 2011), such as decreasing inflammation, oxidation and sequestering oxidized lipids. The best understood and possibly the central mechanism behind many of the beneficial properties of apoA-I is based on its ability to increase reverse cholesterol transport pathway (Yasuda, Ishida et al., 2010; Yvan-Charvet, Wang et al. 2010), which promotes the removal of excess cholesterol from peripheral cells, such as macrophages, and delivers it to the liver for excretion.

It was recently shown that the ability of HDL in serum to efflux cholesterol from macrophages was, in fact, a better predictor of the atheroprotective effect of HDL than the cholesterol content of HDL (HDL-C) (Khera, Cuchel et al. 2011), the current routine diagnostic test for assessing HDL. One of the first steps in the efflux of cholesterol from cells involves the interaction of apoA-1 with the ABCA1 transporter, followed by a detergent-like extraction step whereby apoA-1 removes cholesterol and phospholipids from cells and forms a small nascent HDL particle (Lund-Katz and Phillips 2010). A key structural motif that is necessary for this process to occur is the presence of an amphipathic alpha helix (Remaley, Thomas et al. 2003; Brewer, Remaley et al. 2004), which enables apoA-I or apolipoprotien mimetic peptides to bind to and remove cholesterol and other lipids from the lipid micro domain created by the ABCA1 transporter on the plasma membrane.

In the absence of any associated phospholipids, apolipoproteins do not as readily form amphipathic alpha helices (Frank and Marcel 2000; D'Souza, Stonik et al. 2010). This is particularly true for short synthetic amphipathic peptides, which largely form random coils when present in aqueous buffers, because water effectively competes with the intermolecular hydrogen bonds that stabilize alpha helices. Whether this less conformational constrained state for apolipoproteins or their mimetic peptides is beneficial or detrimental for their interaction with the ABCA1 transporter in the cholesterol efflux process is not known. The phospholipid packing membrane defects into which amphipathic peptides initially insert is relatively small (Cui, Lyman et al., 2011); therefore, it possible that increasing the helicity of amphathic peptide beyond a certain point may interfere with their efflux ability.

The helicity of synthetic peptides can be increased by chemically blocking the end of peptides (Remaley, Amar et al. 2008) and by making longer peptides and peptides with multiple helices (Remaley, Amar et al. 2008). This increases the cost of making such peptides and would be expected to reduce oral bioavailability of longer peptides. It was recently shown that the chemical modification of peptides with linkers also increases helix formation of peptides and has been used to improve the immunogenicity of synthetic peptide vaccines when the antigenic epitope is present in an alpha helical region of an intact protein (Henchey, Jochim et al. 2008; Kutchukian, Yang et al. 2009). In one instance, this peptide modification involves the covalent attachment of a hydrocarbon chain to two different regions of a peptide so that a cross-link is established, thus promoting the alignment of hydrogen bonds between the carbonyl and amino groups in the peptide backbone and facilitating helix formation. This modification has also been shown to improve the membrane permeability of peptides and makes them resistant to proteolysis (Henchey, Jochim et al. 2008; Kutchukian, Yang et al. 2009; Bhattacharya, Zhang et al. 2008). The effect of this modification on apolipoprotiens or their mimetic peptides on their biological properties has not been described.

Another method for increasing the helicity of apolipoproteins or their mimetic peptides is to complex them with lipids. For example, in the lipid-free state, apoA-I is only about 20% helical, but when associated with lipids, it is over 80% helical (Smith, Pownall et al. 1978). Apolipoproteins and their mimetic peptides are typically pre-complexed with phospholipids in therapeutic formulations (Remaley, Amar et al. 2008). One advantage of reconstituting apolipoprotiens and their mimetic peptides with lipids is that it increases the size of the complex, thus potentially extending the half-life of the peptide in the circulation. The reconstitution with phospholipids also potentially reduces the cytotoxicity of the peptide from non-specific lipid extraction and may enable the peptide to efflux cholesterol by other transporters besides ABCA1, such as ABCG1 and SR-BI, which primarily donate cholesterol phospholipid-rich lipoproteins (Rothblat and Phillips 2010). The reconstitution process of apolipoproteins with phospholipids, however, is relatively complex. Most methods are also not scalable, and the reconstitution process significantly adds to the cost of preparing GMP grade material that is suitable for being used as a therapy in humans.

Hydrocarbon chains similar to the acyl group of phospholipids can be covalently attached to peptides during synthesis (Nestor 2009). When apoA-I is fully lipidated with phospholipid, as in the case of when it is bound to HDL, it loses, however, its ability to interact with the ABCA1 transporter (Rothblat and Phillips 2010). The effect of hydrocarbon chain modification on apolipoprotiens or their mimetic peptides on their biological properties has not been previously described.

There remains, therefore, a need in the art for making short apolipoprotein mimetic peptides with stabilized alpha helices that are effective in promoting cholesterol efflux and active in the other biological properties of these peptides without the need for reconstitution with phospholipids. The present invention meets this need.

### BRIEF SUMMARY OF THE INVENTION

The present disclosure provides chemically modified apolipoprotein mimetic peptides that are useful in the treatment of diseases such as cardiovascular disease and diseases associated with inflammation, ischemia and neurodegeneration. In a first aspect of the present invention, apolipoprotein mimetic peptides that stimulate efflux of cholesterol from cells and mediates the other biological properties of these peptides and are helix stabilized by modification with chemical linkers as described herein are provided. These peptides include helix stabilized peptides corresponding to all known apolipoprotein mimetic peptides. In one embodiment, a hydrocarbon chain is attached to two or more sites on the peptide backbone, resulting in helix stabilization of the peptide. In another embodiment, a hydrocarbon chain is attached to only one site of the peptide, resulting in a peptide that has similar cholesterol efflux and other biological properties of a peptide reconstituted with phospholipids. In a second aspect, the present invention provides methods for making the chemically modified apolipoprotein mimetic peptides and peptidomimetic compounds of the invention. In a third aspect, the present invention provides pharmaceutical formulations of the chemically modified apolipoprotien mimetic peptides and peptidomimetic compounds of the invention. In one embodiment, these formulations are suitable for oral administration. In another embodiment, these formulations are suitable for intravenous administration.

These and other embodiments of the invention are described in the accompanying figures and the detailed description of the invention and examples that follow.

These and other embodiments, features and potential advantages will become apparent with reference to the following description and drawings.

### BRIEF DESCRIPTION OF THE DRAWINGS

FIG. 1 depicts the primary sequence and information regarding the biophysical characterization of illustrative peptides discussed herein. Panel A is a helical net plot of the A10, S1A10 and S2A10 peptides and shows the position of chemical modification with linkers. X1=(R)-a-(7′-octanyl)Ala; X2=(S)-a-(4′ pentenyl)Ala. Panel B shows CD-spectroscopy of peptides at 24° C. Panel C shows CD-spectroscopy of heat denaturation of pep tides monitored at residual ellipticity [q] at 222 nm. (dotted line) A10, (solid line) S1A10, (dashed line) S2A10.

FIG. 2 depicts information relating to peptide susceptibility to proteolysis. Peptides A10, S1A10 and S2A10 at a final concentration of 500 μg/ml were incubated for the indicated times with either pepsin (0.5 μg/ml) in 10% acetic acid (pH 2.0) buffer (Panel A) or with chymotrypsin (0.5 μg/ml) in 10 mM NH₄HCO₃ (pH 7.4) buffer (Panel B), and loss of intact peptide was monitored by MALDI-TOF MS. Results are expressed as normalized area under the curve measurements relative to three internal standards.

FIG. 3 depicts Dimyristoyl phosphatidyl choline (DMPC) vesicle solubilization by the indicated peptides. Peptides A10, S1A10 and S2A10 at a final concentration of 33 μg/mL or phosphate buffer saline (PBS) was incubated with DMPC vehicles (200 μg/mL) and monitored for turbidity at 1 min. intervals.

FIG. 4 depicts the results of testing the effect of the peptides on cholesterol efflux. Peptides A10, S1A10, S2A10, 5A at the indicated concentration were incubated with (Panel A) BHK control cells, (Panel B) ABC-A1 transfected BHK cells, (Panel C) ABC-G1 transfected BHK cells, and (Panel D) SR-B1 transfected BHK cells and monitored for cholesterol efflux. Percent of cholesterol efflux over 18 h is expressed as mean±SD of triplicate determinations.

FIG. 5 depicts helicity of peptides as measured by CD-spectroscopy at 24° C.: (dotted line) 5A, (dashed line) acyl-5A.

FIG. 6 depicts the results of testing the effect of peptides on cholesterol efflux. Acyl-5A (Panel A) or 5A (Panel B) at the indicated concentration were incubated with BHK control cells, ABC-A1 transfected BHK cells, ABC-G1 transfected BHK cells, and SR-B1 transfected BHK cells and monitored for cholesterol efflux. Percent of cholesterol efflux over 18 h is expressed as mean±SD of triplicate determinations.

FIG. 7 depicts the results of testing acyl-5A on scrum lipid values in mice. Acyl-5A was injected intravenously in CS7BL/6 mice (n=4) at a dose of 30 mg/kg in saline, and serum was measured for total cholesterol (Panel A), cholesteryl esters (Panel B), and free cholesterol (Panel C) at the indicated, time points. Mean of results are expressed as % of baseline.

FIG. 8 depicts the results of testing the peptide S2A10 on atherosclerosis progression in a mouse model of atherosclerosis. To test the ability of the stapled peptide variant of the last helix of apoA-T containing the long linker, S2A10, it was added to the drinking water (200 ug/mL) of apoE^{hyp}x SR-BI Ko mice. The mice were fed a high fat (21%) diet for 2-weeks and examined for aortic atherosclerosis by en face analysis. As can be seen by there was more than a 2-fold decrease in % area coverage by atherosclerotic plaques, thus demonstrating the efficacy of the oral delivery of stapled variants of apoA-1 mimetic peptides in reducing atherosclerosis.

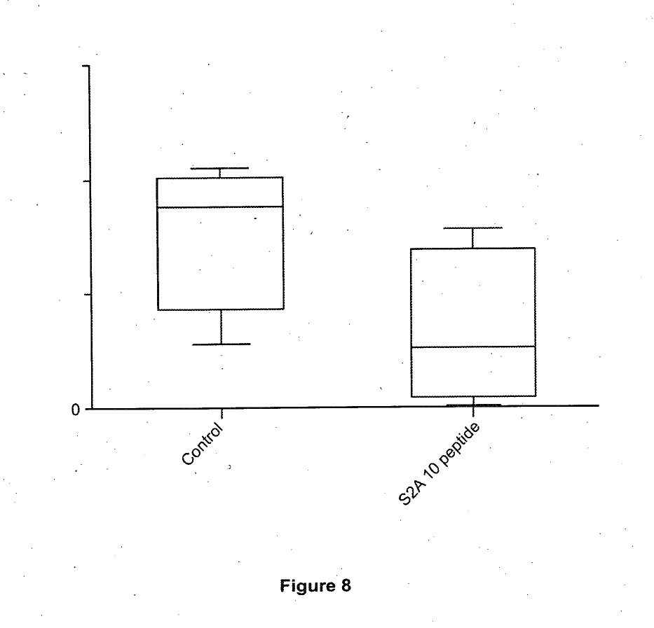

### DETAILED DESCRIPTION OF THE INVENTION

Apolipoproteins are proteins that bind lipids and transport the lipids through the lymphatic and circulatory systems. There are six classes of apolipoproteins and several sub-classes: A (Apo AI, Apo A-II, Apo A-IV and Apo A-V), B (Apo B48 and Apo B100), C (Apo C-I, Apo C-II, Apo C-III and Apo C-IV), D, E and H.

Apolipoproteins can be divided into two categories based on three dimensional and functional differences. Apolipoprotein B, for example, forms low-density lipoprotein (“bad cholesterol”) particles. These proteins have mostly beta-sheet structure and associate with lipid droplets irreversibly.

Other apolipoproteins form high-density lipoprotein (HDL) (“good cholesterol”) particles. These proteins contain alpha-helices and associate with lipid droplets reversibly. HDL apolipoproteins remove cellular cholesterol and phospholipids by a cholesterol-inducible active transport process mediated by a cell membrane protein called ATP-binding cassette transporter A1 (ABCA1).

The last helix of apoA-I has been shown to be critical in the ability of the full-length protein to promote cholesterol efflux, but when synthesized as a single helical peptide, it is unable to promote cholesterol efflux (Panagotopulos, Witting et al. 2002). As shown herein hydrocarbon chain stapling of the last helix of apoA-I results in improved cholesterol efflux from cells relative to unstapled their unstapled counterparts.

Thus, in some embodiments, novel stapled apolipoprotein mimetic peptides that can mimic the function of an apolipoprotein are disclosed. Stapling provides a constraint on a secondary structure, such as an alpha-helical structure.

A “mimetic peptide,” mimic or “peptidomimetic” means a mimetic of a peptide which includes some alteration of the normal peptide chemistry. Peptidomimetics typically enhance some property of the original peptide, such as increase stability, increased efficacy, enhanced delivery, increased half life, etc. Methods of making peptidomimetics based upon a known polypeptide sequence is described, for example, in U.S. Pat. Nos. 5,631,280; 5,612,895; and 5,579,250.

Use of peptidomimetics can involve the incorporation of a non-amino acid residue with non-amide linkages at a given position. One embodiment of the present invention is a peptidomimetic wherein the compound has a bond, a peptide backbone or an amino acid component replaced with a suitable mimic. Some non-limiting examples of unnatural amino acids which may be suitable amino acid mimics include β-alanine, L-α-amino butyric acid, L-γ-amino butyric acid, L-α-amino isobutyric acid, L-ε-amino caproic acid, 7-amino heptanoic acid, L-aspartic acid, L-glutamic acid, N-ε-Boc-N-α-CBZ-L-lysine, N-ε-Boc-N-α-Fmoc-L-lysine, L-methionine sulfone, L-norleucine, L-norvaline, N-α-Boc-N-δCBZ-L-ornithine, N-δ-Boc-N-α-CBZ-L-ornithine, Boc-p-nitro-L-phenylalanine, Boc-hydroxyproline, and Boc-L-thioproline.

In some embodiments, two or more hydrocarbon chain staples are used to stabilize the helicity of peptides.

“Stapled,” “Stapling” and “hydrocarbon-stapling” refer to the introduction into a peptide of at least two moieties capable of undergoing reaction to promote carbon-carbon bond formation when contacted with a reagent to generate at least one cross-linker between the at least two moieties.

“Peptide” or “Polypeptide” refers to any oligopeptide, polypeptide, gene product, expression product, or protein. A polypeptide is comprised of consecutive amino acids. The term “polypeptide” encompasses naturally occurring or synthetic molecules. The term “polypeptide” refers to amino acids joined to each other by peptide bonds or modified peptide bonds, e.g., peptide isosteres, etc. and may contain modified amino acids other than the 20 gene-encoded amino acids.

In some embodiments, the peptide is amino acids 8-33 of ApoA-I (SEQ ID NO:1). In some embodiments, the peptide is amino acids 44-65 of ApoA-I (SEQ ID NO:2). In some embodiments, the peptide is amino acids 66-87 of ApoA-I (SEQ ID NO:3). In some embodiments, the peptide is amino acids 88-98 of ApoA-I (SEQ ID NO:4). In some embodiments, the peptide is amino acids 99-120 of ApoA-I (SEQ ID NO:5). In some embodiments, the peptide is amino acids 121-142 of ApoA-I (SEQ ID NO:6). In some embodiments, the peptide is amino acids 143-164 of ApoA-I (SEQ ID NO:7). In some embodiments, the peptide is amino acids 165-183 of ApoA-I (SEQ ID NO:8). In some embodiments, the peptide is amino acids 187-208 of ApoA-I (SEQ ID NO:9). In some embodiments, the peptide is amino acids 209-219 of ApoA-I (SEQ ID NO:10). In some embodiments, the peptide is amino acids 220-243 of ApoA-I (SEQ ID NO:11). In some embodiments, the peptide is amino acids 7-30 of ApoA-II (SEQ ID NO:12). In some embodiments, the peptide is amino acids 39-50 of ApoA-II (SEQ ID NO:13). In some embodiments, the peptide is amino acids 51-71 of ApoA-II (SEQ ID NO:14). In some embodiments, the peptide is amino acids 7-31 of ApoA-IV (SEQ ID NO:15). In some embodiments, the peptide is amino acids 40-61 of ApoA-IV (SEQ ID NO:16). In some embodiments, the peptide is amino acids 7-32 of ApoC-I (SEQ ID NO:17). In some embodiments, the peptide is amino acids 33-53 of ApoC-I (SEQ ID NO:18). In some embodiments, the peptide is amino acids 28-49 of ApoC-III (SEQ ID NO:19). In some embodiments, the peptide is amino acids 158-182 of ApoE (SEQ ID NO:20). In some embodiments, the peptide is amino acids 26-48 of ApoE (SEQ ID NO:21). In some embodiments, the peptide is amino acids 249-266 of ApoE (SEQ ID NO:22). In some embodiments, the peptide is a synthetic consensus peptide (SEQ ID NO:23).

The term “amino acid” refers to a molecule containing both an amino group and a carboxyl group. Amino acids include alpha-amino acids and beta-amino acids. Suitable amino acids include, without limitation, natural alpha-amino acids such as D- and L-isomers of the 20 common naturally occurring alpha-amino acids (A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q glutamine; R, arginine; S, serine; T, threonine; V, valine; W, tryptophan; Y, tyrosine; Z, glutamine or glutamic acid), unnatural alpha-amino acids, natural beta-amino acids (e.g., beta-alanine), and unnatural beta-amino acids.

In some embodiments, the present invention provides amphipathic apolipoprotein stapled peptides and peptidomimetics in which one or more amino acids is a D amino acid or a non-naturally occurring amino acid or amino acid mimetic.

One or more of the amino acids in a peptide or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A peptide or polypeptide may also be a single molecule or may be a multi-molecular complex, such as a protein. A peptide or polypeptide may be just a fragment of a naturally occurring protein or peptide. A peptide or polypeptide may be naturally occurring, recombinant, or synthetic, or any combination thereof. As used herein “dipeptide” refers to two covalently linked amino acids.

Amino acids used in the construction of peptides of the present invention may be prepared by organic synthesis, or obtained by other routes, such as, for example, degradation of or isolation from a natural source.

There are many known unnatural amino acids any of which may be included in the peptides of the present invention. See for example, S. Hunt, The Non-Protein Amino Acids: In Chemistry and Biochemistry of the Amino Acids, edited by G. C. Barrett, Chapman and Hall, 1985. Some examples of unnatural amino acids are 4-hydroxyproline, desmosine, gamma-aminobutyric acid, beta-cyanoalanine, norvaline, 4-(E)-butenyl-4(R)-methyl-N-methyl-L-threonine, N-methyl-L-leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-1-methylpyrrole-2-carboxylic acid, 2,4-diaminobutyric acid, 2,3-diaminopropionic acid, 2,4-diaminobutyric acid, 2-aminoheptanedioic acid, 4-(aminomethypbenzoic acid, 4-aminobenzoic acid, ortho-, meta- and para-substituted phenylalanines (e.g., substituted with —C(═O)C₆H₅; —CF₃; —CN; -halo; —NO₂; CH₃), disubstituted phenylalanines, substituted tyrosines (e.g., further substituted with —C(═O)C₆H₅; —CF₃; —CN; -halo; —NO₂; CH₃), and statine. Additionally, the amino acids suitable for use in the present invention may be derivatized to include amino acid residues that are hydroxylated, phosphorylated, sulfonated, acylated, and glycosylated, to name a few.

The term “amino acid side chain” refers to a group attached to the alpha- or beta-carbon of an amino acid. A “suitable amino acid side chain” includes, for example, methyl (as the alpha-amino acid side chain for alanine is methyl), 4-hydroxyphenylmethyl (as the alpha-amino acid side chain for tyrosine is 4-hydroxyphenylmethyl) and thiomethyl (as the alpha-amino acid side chain for cysteine is thiomethyl), etc.

A “terminally unsaturated amino acid side chain” refers to an amino acid side chain bearing a terminal unsaturated moiety, such as a substituted or unsubstituted, double bond (e.g., olefinic) or a triple bond (e.g., acetylenic), that participates in crosslinking reaction with other terminal unsaturated moieties in the polypeptide chain. In certain embodiments, a “terminally unsaturated amino acid side chain” is a terminal olefinic amino acid side chain. In certain embodiments, a “terminally unsaturated amino acid side chain” is a terminal acetylenic amino acid side chain. In certain embodiments, the terminal moiety of a “terminally unsaturated amino acid side chain” is not further substituted.

In accordance with the invention, hydrocarbon stapling of amphipathic peptides increases their helical content, reduces their susceptibility to proteolysis and increases their ability to promote cholesterol efflux by the ABCA1 transporter.

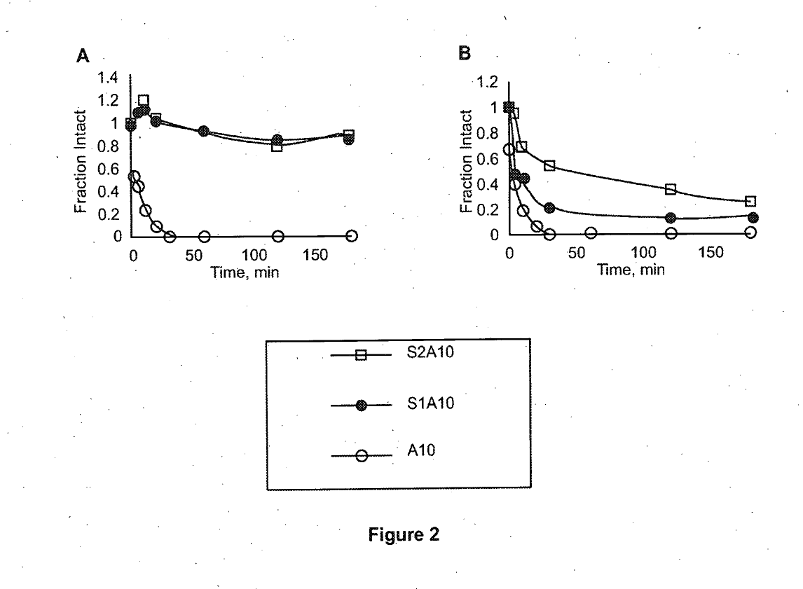

When in a random coil, the chiral carbon of amino acids are on average about 7 angstroms apart, but when peptides form alpha helices, there are approximately 3.5 residues per turn with a mean distance of 1.5 angstroms between each chiral carbon (Henchey, Jochim et al. 2008). The predicted size of the short hydrocarbon linker used to covalently join adjacent turns of an alpha helix for the S1A10 peptide is approximately 3.5 angstroms, thus by introducing this spatial constraint, S1A10 more readily formed an alpha-helix (FIG. 2). A longer hydrocarbon linker, corresponding to the distance between three helices was used to stabilize the S2A10 helix. Thus, in some embodiments, the peptides of the invention are characterized in having linkers that range from 3 to 11 angstroms, i.e., 3.5, 7, and 10.5 angstroms. In addition, while hydrocarbon linkers are typically used, other biologically compatible and similarly stable linkers can be employed.

The phrase “substantially alpha-helical” refers to a polypeptide adopting, on average, backbone (ρ, φ) dihedral angles in a range from about (−90°, −15°) to about (−35°, −70°). Alternatively, the phrase “substantially alpha-helical” refers to a polypeptide adopting dihedral angles such that the φ dihedral angle of one residue and the p dihedral angle of the next residue sums, on average, about −80° to about −125°. In certain embodiments, the polypeptide adopts dihedral angles such that the φ dihedral angle of one residue and the ρ dihedral angle of the next residue sums, on average, about −100° to about −110°. In certain embodiments, the polypeptide adopts dihedral angles such that the φ dihedral angle of one residue and the ρ dihedral angle of the next residue sums, on average, about −105°. Furthermore, the phrase “substantially alpha-helical” may also refer to a polypeptide having at least 50%, 60%, 70%, 80%, 90%, or 95% of the amino acids provided in the polypeptide chain in an alpha-helical conformation, or with dihedral angles as specified herein. Confirmation of a polypeptide's alpha-helical secondary structure may be ascertained by known analytical techniques, such as x-ray crystallography, electron crystallography, fiber diffraction, fluorescence anisotropy, circular dichroism (CD), and nuclear magnetic resonance spectroscopy.

One method of producing the disclosed polypeptides is to link two or more amino acid residues, peptides or polypeptides together by protein chemistry techniques. For example, peptides or polypeptides are chemically synthesized using currently available laboratory equipment using either Fmoc (9-fluorenylmethyloxycarbonyl) or Boc (tert-butyloxycarbonoyl) chemistry (Applied Biosystems, Inc., Foster City, Calif.). A peptide or polypeptide can be synthesized and not cleaved from its synthesis resin, whereas the other fragment of a peptide or protein can be synthesized and subsequently cleaved from the resin, thereby exposing a terminal group, which is functionally blocked on the other fragment. By peptide condensation reactions, these two fragments can be covalently joined via a peptide bond at their carboxyl and amino termini, respectively, (Grant G A (1992) Synthetic Peptides: A User Guide. W.H. Freeman and Co., New York (1992); Bodansky M and Trost B., Ed. (1993) Principles of Peptide Synthesis. Springer-Verlag Inc., New York).

Non-natural amino acids containing olefin-bearing tethers may be synthesized, for example, according to methodology provided in Schafineister et al. and Williams and Im (J. Am. Chem. Soc., 113:9276-9286 (1991)).

Alternatively, the peptide or polypeptide is independently synthesized in vivo. Once isolated, these independent peptides or polypeptides may be linked to form a peptide or fragment thereof via similar peptide condensation reactions.

In some embodiments, one or more pairs of α,α-disubstituted non-natural amino acids containing olefin-bearing tethers corresponding to the native amino acids are substituted into the alpha-helices of the Apo derived peptide, as depicted in Table 1. In other embodiments, one or more pairs of α,α-disubstituted non-natural amino acids containing olefin-bearing tethers corresponded to the native amino acids are substituted into residues as depicted in Table 1.

In other embodiments, other types of linkers (Henchey, Jochim et al. 2008) that stabilize the helicity of peptides are used. Suitable linkers include, without limitation, disulfides, lactams, metal mediated bridges, hydrazones, photoclick staples, cysteine staples and hydrogen bond surrogates or alternative amino acids (Henchey, Jochim et al. 2008), such as beta amino acids that promote helix formation.

In some embodiments, polar or charged linkers placed on the hydrophilic face of amphipathic apolipoprotein peptides and peptidomimetics that stabilize the helicity of peptides are employed in the peptides of the invention.

In other embodiments, other proteins besides apoA-J or synthetic peptides that contain amphipathic helices (Table 1) can be modified by chemical linkers for promoting cholesterol efflux, as well as the other biological properties of these peptides. For example, the ligand binding domains of apoE and apoB are in helical regions and promote the uptake of lipoproteins by various receptors, such as the LDL-receptor. Stabilization of the ligand binding domain of these peptides by hydrocarbon chain linkers and other types of linkers, increases the uptake of lipoproteins by cellular receptors when peptides with these modifications are associated with lipoproteins. Helical regions on apolipoproteins also act as docking sites or regulators of many different lipoprotein modifying proteins, such as Cholesteryl Ester Transfer protein, phospholipid transfer protein, lecithin:choleserol acyltransferase, lipoprotein lipase, endothelial lipase, hepatic lipase plus others. Stabilization of helical regions of apolipoproteins or their short synthetic peptide mimics by chemical linkers can also be used for promoting the interaction with these other proteins.

In other embodiments, other apolipoprotein mimetic peptides, such as the ones shown in Table 1 or peptidomimetics are provided by the invention in a modified from, resulting from covalent attachment of a hydrocarbon chain, as described herein.

In other embodiments, apolipoprotein mimetic peptides and peptidomimetics are provided by the invention in a modified form, resulting from covalent attachment of different hydrocarbon chains of either shorter (ex. Capric acid or Lauric acid) or longer length (ex. Palmitic acid and Stearic Acid) and either fully saturated (ex. Palmitc acid and Stearic acid) or unsaturated (ex. Linolenic acid or Arachidonic acid) in either the trans or cis configuration.

In other embodiments, apolipoprotein mimetic peptides and peptidomimetics are provided by the invention in a modified form, resulting from covalent attachment of a hydrocarbon chain with one of a variety of chemical bonds, such as ester bonds, ether bonds, amide bonds or by direct incorporation with FMOC-amino acid derivatives containing a hydrocarbon chain, such as the ones described herein.

In other embodiments, apolipoprotein mimetic peptides and peptidomimetics are provided by the invention in a modified form, resulting from covalent attachment of one or more hydrocarbon chains at the amino terminal end, the carboxy terminal end or any intervening site on peptides.

As used herein, “substantially pure” means that the depicted or named compound is at least about 60% by weight. For example, “substantially pure” can mean about 60%, 70%, 72%, 75%, 77%, 80%, 82%, 85%, 86%, 87%, 88%, 89%, 90%, 91%, 92%, 93%, 94%, 95%, 96%, 97%, 98%, 99%, 99.5%, 99.9%, or a percentage between 70% and 100%. In one embodiment, substantially pure means that the depicted or named compound is at least about 75%. In a specific embodiment, substantially pure means that the depicted or named compound is at least about 90% by weight.

The chemically modified peptides and peptide analogs of the disclosure can be used alone or in combination therapy with other lipid lowering compositions or drugs used to treat the foregoing conditions. Such therapies include, but are not limited to simultaneous or sequential administration of the drugs involved. For example, in the treatment of hypercholesterolemia or atherosclerosis, the multidomain peptide or peptide analog formulations can be administered with anyone or more of the cholesterol lowering therapies currently in use, for example, bile-acid resins, niacin and statins. In another embodiment, they can be used in conjunction with statins or fibrates to treat hyperlipidemia, hypercholesterolemia and/or cardiovascular disease, such as atherosclerosis. In yet another embodiment, they can be used in combination with an anti-microbial agent and/or an anti-inflammatory agent.

The chemically modified peptides and peptide analogs of the disclosure can be used to treat any disorder in animals, especially mammals (e.g., humans), for which promoting lipid efflux is beneficial, as well as the other biological properties of HDL, such as increasing endothelial cell integrity, anti-inflammation, antithrombosis, and anti-oxidation. Such conditions include, but are not limited to, hyperlipidemia (e.g., hypercholesterolemia), cardiovascular disease (e.g., atherosclerosis), restenosis (e.g., atherosclerotic plaques), peripheral vascular disease, acute coronary syndrome, reperfusion myocardial injury, and the like. They can also be used during the treatment of thrombotic and ischemic stroke and during thrombolytic treatment of occluded coronary artery disease and Alzheimer's disease.

In some embodiments, one or more of the chemically modified peptides and peptide analogs of the disclosure are administered, in the “native” form or, if desired, in the form of salts, esters, amides, prodrugs, derivatives, provided the salt, ester, amide, prodrug or derivative is suitable pharmacologically, i.e., effective in the present method. Salts, esters, amides, prodrugs and other derivatives of the disclosed agents can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

For example, acid addition salts are prepared from the free base using conventional methodology, that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Particularly preferred acid addition salts of the active agents herein are halide salts, such as may be prepared using hydrochloric or hydrobromic acids. Conversely, preparation of basic salts of the active agents of this invention are prepared in a similar manner using a pharmaceutically acceptable base such as sodium hydroxide, potassium hydroxide, ammonium hydroxide, calcium hydroxide, trimethylamine, or the like. Particularly preferred basic salts include alkali metal salts, e.g., the sodium salt, and copper salts.

Preparation of Esters Typically Involves Functionalization of Hydroxyl and/or carboxyl groups which may be present within the molecular structure of the drug. The esters are typically acyl-substituted derivatives of free alcohol groups, i.e., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides and prodrugs can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety that results in a compound that is therapeutically inactive until modified by an individual's metabolic system.

The chemically modified peptides and peptide analogs of the disclosure are useful for oral, parenteral, topical, nasal (or otherwise inhaled), rectal, or local administration, such as by aerosol or transdermally, for prophylactic and/or therapeutic treatment of one or more of the pathologies/indications described herein (e.g., atherosclerosis and/or eye disease and/or symptoms thereof). The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectables, implantable sustained-release formulations, lipid complexes, etc.

The chemically modified peptides and peptide analogs of the disclosure are typically combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agents, or excipients or other stabilizers and/or buffers.

Other physiologically acceptable compounds include wetting agents, emulsifying, agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s).

The excipients are preferably sterile and generally free of undesirable matter. These compositions may be sterilized by conventional, well-known sterilization techniques.

In therapeutic applications, the chemically modified peptides and peptide analogs of the disclosure are administered to a patient suffering from one or more symptoms of the one or more pathologies described herein, or at risk for one or more of the pathologies described herein in an amount sufficient to prevent and/or cure and/or or at least partially prevent or arrest the disease and/or its complications. An amount adequate to accomplish this is defined as a “therapeutically effective dose.” Amounts effective, for this use will depend upon the severity of the disease and the general state of the patient's health. Single or multiple administrations of the compositions may be administered depending on the dosage and frequency as required and tolerated by the patient. In any event, the composition should provide a sufficient quantity of the active agents of the formulations of this invention to effectively treat (ameliorate one or more symptoms) the patient.

The concentration of the chemically modified peptides and peptide analogs of the disclosure can be selected primarily based on fluid volumes, viscosities, body weight in accordance with the particular mode of administration selected and the patient's needs. Concentrations, however, will typically be selected to provide dosages ranging from about 0.1 or 1 mg/kg/day to about 50 mg/kg/day and sometimes higher. Typical dosages range from about 3 mg/kg/day to about 3.5 mg/kg/day, preferably from about 3.5 mg/kg/day to about 7.2 mg/kg/day, more preferably from about 7.2 mg/kg/day to about 11.0 mg/kg/day, and most preferably from about 11.0 mg/kg/day to about 15.0 mg/kg/day. In certain preferred embodiments, dosages range from about 10 mg/kg/day to about 50 mg/kg/day. In certain embodiments, dosages range from about 20 mg to about 50 mg given orally twice daily. It will be appreciated that such dosages may be varied to optimize a therapeutic regimen in a particular subject or group of subjects.

In certain preferred embodiments, the active agents of this invention are administered orally (e.g., via a tablet) or as an injectable in accordance with standard methods well known to those of skill in the art. In other preferred embodiments, the peptides, may also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

Other preferred formulations for topical drug delivery include, but are not limited to, ointments and creams. Ointments are semisolid preparations which are typically based on petrolatum or other petroleum derivatives. Creams containing the selected active agent, are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

In addition, the chemically modified peptides and peptide analogs of the disclosure can be administered via intraocular injection (e.g., intravitreal injection) in accordance with standard methods well known to those of skill in the art.

Unlike typical peptide formulations, the peptides of this invention comprising D-form amino acids can be administered, even orally, without protection against proteolysis by stomach acid, etc. Nevertheless, in certain embodiments, peptide delivery can be enhanced by the use of protective excipients. This is typically accomplished either by complexing the polypeptide with a composition to render it resistant to acidic and enzymatic hydrolysis or by packaging the polypeptide in an appropriately resistant carrier such as a liposome. Means of protecting polypeptides for oral delivery are well known in the art (see, e.g., U.S. Pat. No. 5,391,377 describing lipid compositions for oral delivery of therapeutic agents).

Elevated serum half-life can be maintained by the use of sustained-release protein “packaging” systems. Such sustained release systems are well known to those of skill in the art. In one preferred embodiment, the ProLease biodegradable microsphere delivery system for proteins and peptides (Tracy (1998) Biotechnol. Prog., 14: 108; Johnson et al. (1996) Nature Med. 2: 795; Herbert et al. (1998), Pharmaceut. Res. 15, 357) a dry powder composed of biodegradable polymeric microspheres containing the active agent in a polymer matrix that can be compounded as a dry formulation with or without other agents.

The ProLease microsphere fabrication process was specifically designed to achieve a high encapsulation efficiency while maintaining integrity of the active agent. The process consists of (i) preparation of freeze-dried drug particles from bulk by spray freeze-drying the drug solution with stabilizing excipients, (ii) preparation of a drug-polymer suspension followed by sonication or homogenization to reduce the drug particle size, (iii) production of frozen drug-polymer microspheres by atomization into liquid nitrogen, (iv) extraction of the polymer solvent with ethanol, and (v) filtration and vacuum drying to produce the final dry-powder product. The resulting powder contains the solid form of the active agents, which is homogeneously and rigidly dispersed within porous polymer particles. The polymer most commonly used in the process, poly(lactide-co-glycolide) (PLG), is both biocompatible and biodegradable.

Encapsulation can be achieved at low temperatures (e.g., −40° C.). During encapsulation, the protein is maintained in the solid state in the absence of water, thus minimizing water-induced conformational mobility of the protein, preventing protein degradation reactions that include water as a reactant, and avoiding organic-aqueous interfaces where proteins may undergo denaturation. A preferred process uses solvents in which most proteins are insoluble, thus yielding high encapsulation efficiencies (e.g., greater than 95%).

In another embodiment, one or more components of the solution can be provided as a “concentrate”, e.g., in a storage container (e.g., in a premeasured volume) ready for dilution, or in a soluble capsule ready for addition to a volume of water.

The foregoing formulations and administration methods are intended to be illustrative and not limiting. It will be appreciated that, using the teaching provided herein, other suitable formulations and modes of administration can be readily devised.

In some embodiments, the disclosed agents are administered in conjunction with one or more lipids. The lipids can be formulated as are excipient to protect and/or enhance transport/uptake of the agents or they can be administered separately.

The lipids can be formed into liposomes that encapsulate the active agents of this invention and/or they can be complexed/admixed with the active agents and/or they can be covalently coupled to the active agents. Methods of making liposomes and encapsulating reagents are well known to those of skill in the art (see, e.g., Martin and Papahadjopoulos (1982) J. Biol. Chem., 257: 286-288; Papahadjopoulos et al. (1991) Proc. Natl. Acad. Sci. USA, 88: 11460-11464; Huang et al. (1992) Cancer Res., 52:6774-6781; Lasic et al. (1992) FEBS Lett., 312: 255-258., and the like).

Preferred phospholipids for use in these methods have fatty acids ranging from about 4 carbons to about 24 carbons in the sn-1 and sn-2 positions. In certain preferred embodiments, the fatty acids are saturated. In other preferred embodiments, the fatty acids can be unsaturated.

The fatty acids in these positions can be the same or different. Particularly preferred phospholipids have phosphorylcholine at the sn-3 position.

In certain embodiments the chemically modified peptides and peptide analogs of the disclosure are contained within biocompatible matrices (e.g. biocompatible polymers such as urethane, silicone, and the like). Suitable biocompatible materials are described, for example, in U.S. Patent Publications 2005/0084515, 2005/00791991, 2005/0070996, and the like which are incorporated herein by reference. In various embodiments the polymers include, but are not limited to silicone-urethane copolymer, a polyurethane, a phenoxy, ethylene vinyl acetate, polycaprolactone, poly(lactide-co-glycolide), polylactide, polysulfone, elastin, fibrin, collagen, chondroitin sulfate, a biocompatible polymer, a biostable polymer, a biodegradable polymer.

Thus, in certain embodiments this invention provides a stent for delivering drugs to a vessel in a body. The stent typically comprises stent framework including a plurality of reservoirs formed therein. The reservoirs typically include an active agent and/or active agent-containing polymer positioned in the reservoir and/or coated on the surface of the stent. In various embodiments the stent is a metallic base or a polymeric base. Certain preferred stent materials include, but are not limited to stainless steel, nitinol, tantalum, MP35N alloy, platinum, titanium, a suitable biocompatible alloy, a suitable biocompatible polymer, and/or a combination thereof.

In various embodiments where the stent comprises pores (e.g. reservoirs), the pores can include micropores (e.g., having a diameter that ranges from about 10 to about 50 μm, preferably about 20 μm or less). In various embodiments the micropores have a depth in the range of about 10 μm to about 50 μm. In various embodiments the micropores extend through the stent framework having an opening on an interior surface of the stent and an opening on an exterior surface of the stent. In certain embodiments the stent can, optionally comprise a cap layer disposed on the interior surface of the stent framework, the cap layer covering at least a portion of the through-holes and providing a barrier characteristic to control an elution rate of the active agent(s) in the polymer from the interior surface of the stent framework. In various embodiments the reservoirs comprise channels along an exterior surface of the stent framework. The stent can optionally have multiple layers of polymer where different layers of polymer carry different active agent(s) and/or other drugs.

In certain embodiments the stent comprises: an adhesion layer positioned between the stent framework and the polymer. Suitable adhesion layers include, but are not limited to a polyurethane, a phenoxy, poly(lactide-co-glycolide)-, polylactide, polysulfone, polycaprolactone, an adhesion promoter, and/or a combination thereof.

In addition to stents, the active agents can be coated on or contained within essentially any implantable medical device configured for implantation in a extravascular and/or intravascular location.

Also provided are methods of manufacturing a drug-polymer stent, comprising. The methods involve providing a stent framework; cutting a plurality of reservoirs in the stent framework, e.g., using a high power laser; applying one or more of the active agents and/or a drug polymer to at least one reservoir; drying the drug polymer; applying a polymer layer to the dried drug polymer; and drying the polymer layer. The active agent(s) and/or polymer(s) can be applied by any convenient method including but not limited to spraying, dipping, painting, brushing and dispensing.

Also provided are methods of treating a vascular condition and/or a condition characterized by an inflammatory response and/or a condition characterized by the formation of oxidized reactive species. The methods typically involve positioning a stent or other implantable device as described above within the body (e.g. within a vessel of a body) and eluting at least active agent from at least one surface of the implant.

In certain embodiments, one or more active agents described herein are administered alone or in combination with other therapeutics as described herein in implantable (e.g., subcutaneous) matrices.

A drawback with standard drug dosing is that typical delivery of drugs results in a quick burst of medication at the time of dosing, followed by a rapid loss of the drug from the body. Most of the side effects of a drug occur during the burst phase of its release into the bloodstream. Secondly, the time the drug is in the bloodstream at therapeutic levels is very short, most is used and cleared during the short burst.

Drugs (e.g., the active agents described herein) imbedded in various matrix materials for sustained release provides some solution to these problems. Drugs embedded, for example, in polymer beads or in polymer wafers have several advantages. First, most systems allow slow release of the drug, thus creating a continuous dosing of the body with small levels of drug. This typically prevents side effects associated with high burst levels of normal injected or pill based drugs. Secondly, since these polymers can be made to release over hours to months, the therapeutic span of the drug is markedly increased. Often, by mixing different ratios of the same polymer components, polymers of different degradation rates can be made, allowing remarkable flexibility depending on the agent being used. A long rate of drug release is beneficial for people who might have trouble staying on regular dosage, such as the elderly, but is also an ease of use improvement that everyone can appreciate. Most polymers can be made to degrade and be cleared by the body over time, so they will not remain in the body after the therapeutic interval.

Another advantage of polymer based drug delivery is that the polymers often can stabilize or solubilize proteins, peptides, and other large molecules that would otherwise be unusable as medications. Finally, many drug/polymer mixes can be placed directly in the disease area, allowing specific targeting of the medication where it is needed without losing drug to the “first pass” effect. This is certainly effective for treating the brain, which is often deprived of medicines that can't penetrate the blood/brain barrier.

A number of implantable matrix (sustained release) systems are known to those of skill and can readily be adapted for use with one or more of the active agents described herein. Suitable sustained release systems include, but are not limited to Re-Gel®, SQ2Gel®, and Oligosphere® by MacroMed, ProLease® and Medisorb® by Alkermes, Paclimer® and Gliadel® Wafer by Guilford pharmaceuticals, the Duros implant by Alza, acoustic bioSpheres by Point Biomedical, the Intelsite capsule by Scintipharma, Inc., and the like.

Other “specialty” delivery systems include, but are not limited to lipid based oral mist that allows absorption of drugs across the oral mucosa, developed by Generex Biotechnology, the oral transmucosal system (OTS™) by Anesta Corp., the inhalable dry powder and PulmoSpheres technology by Inhale Therapeutics, the AERx® Pulmonary Drug Delivery System by Aradigm, the AIR mechanism by Alkermes.

Another approach to delivery developed by Alkermes is a system targeted for elderly and pediatric use, two populations for which taking pills is often difficult is known as Drug Sipping Technology (DST). The medication is placed in a drinking straw device, prevented from falling out by filters on either end of it. The patient merely has to drink clear liquid (water, juice, soda) through the straw. The drug dissolves in the liquid as it is pulled through and is ingested by the patient. The filter rises to the top of the straw when all of the medication is taken. This method has the advantage in that it is easy to use, the liquid often masks the medication's taste, and the drug is pre-dissolved for more efficient absorption.

It is noted that these uses and delivery systems are intended to be illustrative and not limiting. Using the teachings provided herein, other uses and delivery systems will be known to those of skill in the art.

The chemically modified peptides and peptide analogs of the disclosure can be co-administered with other agents, such as niclosamide, which have been shown to further prevent proteolysis and enhance absorption of amphipathic peptides (Navab, Ruchala et al. 2009).

In various embodiments, the use of combinations of two or more agents described is contemplated in the treatment of the various pathologies/indications described herein. The use of combinations of active agents can alter pharmacological activity and bioavailability.

In certain embodiments this invention thus contemplates combinations of, for example, these three peptides to reduce the amount to reduce production expense, and/or to optimize dosage regimen, therapeutic profile, and the like. In certain embodiments combinations of the active agents described herein can be simply co-administered and/or added together to form a single pharmaceutical formulation. In certain embodiments the various active agent(s) can be complexed together (e.g. via hydrogen bonding) to form active agent complexes that are more effective than the parent agents.

Additional pharmacologically active materials (i.e., drugs) can be delivered in conjunction with one or more of the active agents described herein. In certain embodiments, such agents include, but are not limited to agents that reduce the risk of atherosclerotic events and/or complications thereof. Such agents include, but are not limited to beta blockers, beta blockers and thiazide diuretic combinations, statins, aspirin, ace inhibitors, ace receptor inhibitors (ARBs), and the like.

Thus, in certain embodiments this invention provides methods for enhancing the activity of statins. The methods generally involve administering one or more of the active agents described herein, as described herein in conjunction with one or more statins. The active agents achieve synergistic action between the statin and the agent(s) to ameliorate one or more symptoms of atherosclerosis. In this context statins can be administered at significantly lower dosages thereby avoiding various harmful side effects (e.g., muscle wasting) associated with high dosage statin use and/or the anti-inflammatory properties of statins at any given dose are significantly enhanced.

Suitable statins include, but are not limited to pravastatin (Pravachol/Bristol-Myers Squibb), simvastatin (Zocor/Merck), lovastatin (Mevacor/Merck).

In various embodiments the agent(s) described herein are administered in conjunction with one or more beta blockers. Suitable beta blockers include cardioselective (selective beta 1 blockers), e.g., acebutolol (Sectral™), atenolol (Tenormin™), betaxolol (Kerlone™), bisoprolol (Zebeta™), metoprolol (Lopressor™). Suitable non-selective blockers include without limitation carteolol (Cartrol™), nadolol (Corgard™), penbutolol (Levatol™), pindolol (Visken™), propranolol (Inderal™), timolol (Blockadren™) and labetalol (Normodyne™, Trandate™).

Suitable beta blocker thiazide diuretic combinations include Lopressor HCT, ZIAC, Tenoretic, Corzide, Timolide, Inderal LA 40/25, Inderide, Normozide.

Suitable ace inhibitors include, but are not limited to captopril (e.g. Capotez™ by Squibb), benazepril (e.g., Lotensin™ by Novartis), enalapril (e.g., Vasotec™ by Merck), fosinopril (e.g., Monopril™ by Bristol-Myers), lisinopril (e.g. Prinivil™ by Merck or Zestril™ by Astra-Zeneca), quinapril (e.g. Accupril™ by Parke-Davis), ramipril (e.g., Altace™ by Hoechst Marion Roussel, King Pharmaceuticals), imidapril, perindopril erbumine (e.g., Aceon™ by Rhone-Polenc Rorer) and trandolapril (e.g., Mavik™ by Knoll Pharmaceutical). Suitable ARES (Ace Receptor Blockers) include but are not limited to losartan (e.g. Cozaar™ by Merck), irbesartan (e.g., Avapro™ by Sanofi), candesartan (e.g., Atacand™ by Astra Merck) and valsartan (e.g., Diovan™ by Novartis).

In various embodiments, one or more agents described herein are administered with one or more of the drugs identified below.

Thus, in certain embodiments one or more active agents are administered in conjunction with cholesteryl ester transfer protein (CETP) inhibitors (e.g., torcetrapib, JTT-705. CP-529414) and/or acyl-CoA: cholesterol O-acyltransferase (ACAT) inhibitors (e.g., Avasimibe (CI-1011), CP 113818, F-1394, and the like), and/or immunomodulators (e.g., FTY720 (sphingosine-1-phosphate receptor agonist), Thalomid (thalidomide), Imuran (azathioprine), Copaxone (glatiramer acetate), Certican® (everolimus), Neoral® (cyclosporine), and/or dipeptidyl-peptidase-4 (DPP4) inhibitors (e.g., 2-Pyrrolidinecarbonitrile, 1-[[[2-[(5-cyano-2-pyridinyl)amino]ethyl]amino]acetyl], see also U.S. Patent Publication 2005-0070530), and/or calcium channel blockers (e.g., Adalat, Adalat CC, Calan, Calan SR, Cardene, Cardizem, Cardizem CD, Cardizem SR, Dilacor-XR, DynaCirc, Isoptin, Isoptin SR, Nimotop, Norvasc, Plendil, Procardia, Procardia XL, Vascor, Verelan), and/or peroxisome proliferator-activated receptor (PPAR) agonists for, e.g., α, γ, receptors (e.g., Azelaoyl PAF, 2-Bromohekadecanoic acid, Ciglitizone, Clofibrate, 15-Deoxy-δ^{12,14}-prostaglandin J₂, Fenofibrate, Fmoc-Leu-OH, GW1929, GW7647, 8(S)-Hydroxy-(5Z,9E,11Z,14Z)-eicosatetraenoic acid (8(S)-HETE), Leukotriene B₄, LY-171,883 (Tomelukast), Prostaglandin A₂, Prostaglandin J₂, Tetradecylthioacetic acid (TTA), Troglitazone (CS-045) and WY-14643 (Pirinixic acid).

In certain embodiments one or more of the active agents are administered in conjunction with fibrates (e.g., clofibrate (atromid), gemfibrozil (lopid), fenofibrate (tricor), etc.), bile acid sequestrants (e.g., cholestyramine, colestipol, etc.), cholesterol absorption blockers (e.g., ezetimibe (Zetia), etc.), Vytorin ((ezetimibe/simvastatin combination), and/or steroids, warfarin, and/or aspirin and/or angiotensin II receptor antagonists (e.g., losartan (Cozaar), valsartan (Diovan), irbesartan (Avapro), candesartan (Atacand) and telmisartan (Micardis).

In another embodiment this invention provides kits for amelioration of one or more symptoms of atherosclerosis or for the prophylactic treatment of a subject (human or animal) at risk for atherosclerosis and/or the treatment or prophylaxis of one or more of the conditions described herein.

The kits preferably comprise a container containing one or more of the chemically modified peptides and peptide analogs of the disclosure. The active agent(s) can be provided in a unit dosage formulation (e.g. suppository, tablet, caplet, patch, etc.) and/or may be optionally combined with one or more pharmaceutically acceptable excipients.

The kit can, optionally, further comprise one or more other agents used in the treatment of the condition/pathology of interest. Such agents include, but are not limited to, beta blockers, vasodilators, aspirin, statins, ace inhibitors or ace receptor inhibitors (ARBs) as described above.

In addition, the kits optionally include labeling and/or instructional materials providing directions (i.e., protocols) for the practice of the methods or use of the “therapeutics” or “prophylactics” of this invention. Preferred instructional materials describe the use of one or more active agent(s) of this invention to mitigate one or more symptoms of atherosclerosis (or other pathologies described herein) and/or to prevent the onset or increase of one or more of such symptoms in an individual at risk for atherosclerosis (or other pathologies described herein). The instructional materials may also, optionally, teach preferred dosages/therapeutic regiment, counter indications and the like.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to interne sites that provide such instructional materials.

### EXAMPLES

### Example 1

### Two-Site Attachment of Hydrocarbon Chains to Apolipoprotein Mimetic Peptides

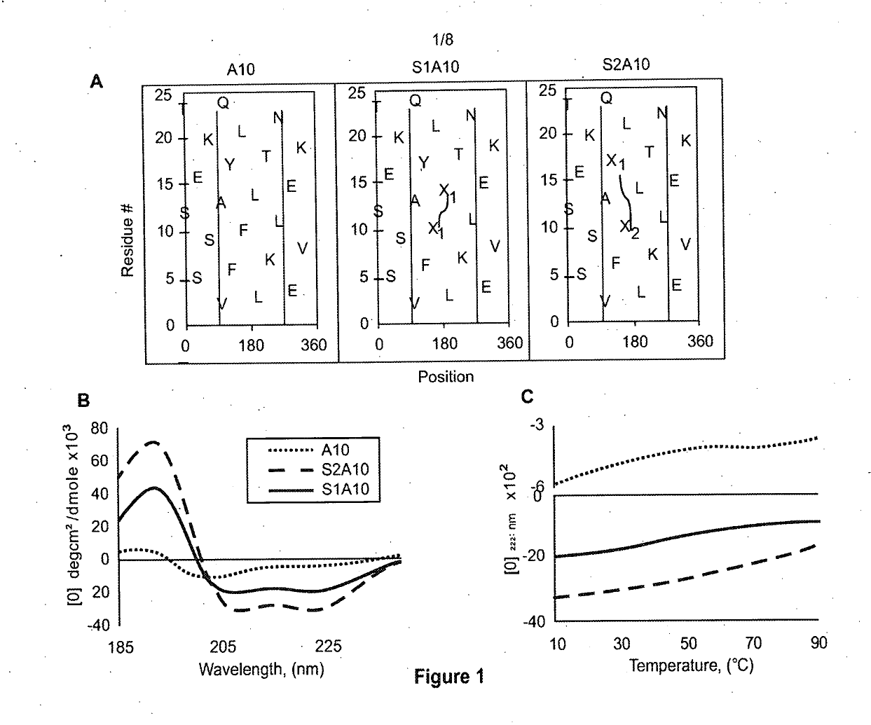

The primary sequence of the last helix of apoA-I, referred to as “A10” (Ac-VLESFKVSFLSALEEYTKKLNTQ-NH2) and the position of the hydrocarbon linkers in the two modified stapled peptides, “S1A10” and “S2A10,” are shown as helical net plots in FIG. 1. Each of these peptides were made by solid phase synthesis, using standard Fmoc chemistry and the Fmoc-modified amino acid linkers ((R)-a-(7′-octanyl)Ala and (S)-a-(4′ pentenyl)Ala) (AnaSpec, Inc.)(Sviridov et al. (2011) Biochemical and Biophysical Res. Comm. 410:446-451; incorporated by reference in its entirety).

To preserve the hydrophobic moment of the modified peptides, the hydrophobic hydrocarbon staples were placed near the center of the hydrophobic region. The cross linking of the modified amino acid linkers was done by the olefin metathesis reaction with Bis(tricyclohexyl-phosphine)-benzyldine ruthenium (IV) dichloride as the catalyst. Schafineister et al (2000) J. Am. Chem. Soc. 122:5891-5892, incorporated by reference in its entirety. In the case of the stapled peptide, S1A10, a hydrocarbon linker was used to covalently bridge two adjacent helices in the center (third and fourth turn) of the peptide. In the case of S2A10, a longer hydrocarbon linker was used for linking the third and fifth helical turn of the peptide.

Each of the peptides was purified by reverse phase HPLC and was determined to be over 95% pure by LC-MS analysis.

The helical nature of the peptides was determined by circular dichroism spectroscopy. Peptides (0.1 mg/mL) in sodium phosphate buffer (pH 7.4) were loaded into a quartz cuvette (d=0.2 cm path length) and CD spectra from 185 to 240 nm were recorded on a Jasco J715 spectropolarimeter at 24° C. Data were normalized by calculating the mean residue ellipticity (θ). Peptide thermostability was monitored at 220 nm by increasing temperature from 10° to 90° C.

Based on CD spectroscopy, the unstapled A10 peptide at 24° C. was 17% helical in aqueous buffer (FIG. 1B), whereas S1A10 and S2A10 were 62% and 97% helical, respectively. By CD spectroscopy (FIG. 1C), the heat denaturation curve for S1A10 was parallel to A10, but S1A10 was more helical than A10 at all temperatures tested and even had some residual helical content at the highest temperature tested, which may possibly be within the cross linked region. The S2A10 peptide, with the longer linker, showed even less susceptibility to thermal denaturation. Helicity at 90° C. was calculated to be 11%, 25%, and 48% for A10, S1A10 and S2A10, respectively.

### Example 2

### Susceptibility of Stapled Peptides to Protease Action

Unfolded proteins that do not have a significant amount of secondary structure, such as alpha helices, are more readily digested by proteases, which limits the oral availability of therapeutic peptides and potentially reducing their half-life in the circulation (Bhattacharya, Zhang et al. 2008). To demonstrate the improved stability and oral availability of the stapled peptides of the invention, the 3 peptides in FIG. 1 were tested for their susceptibility to proteolysis by pepsin and chymotrypsin, which are both abundant digestive tract proteases. Several potential pepsin and/or chymotrypsin cleavage sites are found within and outside the linker region of the peptides A10, S1A10 and S2A10.

A10, S1A10 and S2A10 (5 mg/ml) were diluted 10× with either 10% acetic acid (pH 2), containing pepsin (0.5 μg/ml final) or in 10 mM NH₄HCO₃ (pH 7), containing chymotrypsin (0.5 μg/ml final). After incubation at 37° C., aliquots at various time points were removed and dried on a MALDI Anchor Chip target at 45° C. MALDI standards (Bombesin, ACTH 1-17, ACTH 18-39, Somastatin 28, CHCA in 50% ACN, 0.1% TFA) and matrix were added sequentially and allowed to dry between additions. Samples were analyzed on a Bruker AutoFlex III (Bruker Daltonics) in positive ion reflectron mode. Relative protein concentrations were determined from areas under the curve, using the MALDI standards as calibrators.

After the protease digestions, a decrease in the peaks corresponding to the intact parent peptides were monitored by MALDI-TOF mass spectrometry, as shown in FIG. 2. The A10 peptide was readily digested by both proteases, with less than 5% of intact peptide remaining after 30 min. In contrast, the stapled peptides were relatively resistant to proteolysis by pepsin and chymotrypsin, although S2A10 appeared to be less sensitive to chymotrypsin than S1A10. Both stapled peptides were almost fully digested after longer incubation times, with only 0.4% and 0.6% remaining for S1A10 and 12.2% and 3.8% remaining for S2A10 after digestion for 24 h with pepsin and chymotrypsin, respectively.

### Example 3

### Assessment of the Ability of the Peptides to Act Like Detergents

A phospholipid vesicle solubilization assay was undertaken to assess the ability of the peptides to act like detergents. Dimyristoyl phosphatidyl choline (DMPC) vesicles were prepared by resuspension of dried DMPC with PBS and vortexing for 5 min. Changes in light scattering upon peptide addition were monitored at 24° C. every minute for 1 h at 660 nm, with shaking in a Perkin Elmer plate reader.

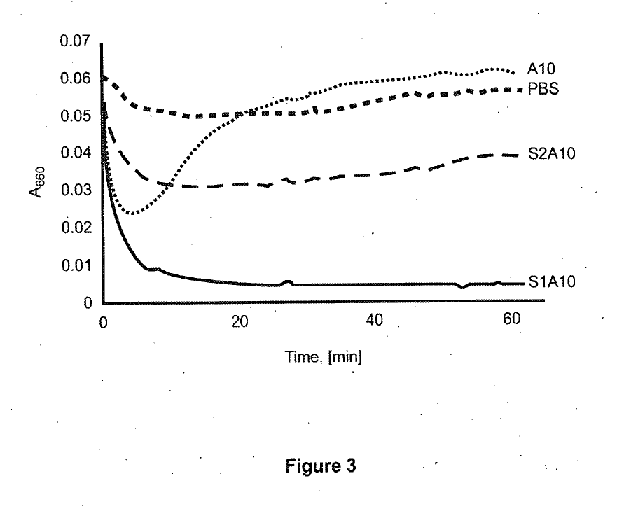

The unstapled A10 peptide caused some initial solubilization of the DMPC vesicles, but at later time points, the turbidity of the solution increased. In contrast, both stapled peptides readily dissolved the phospholipid vehicles (FIG. 3).

### Example 4

### Single-Site Attachment of Hydrocarbon Chains to Apolipoprotein Mimetic Peptides

To illustrate another aspect of the invention, in which single site attachment of a hydrocarbon chain to a peptide is used to improve its properties, a 40-amino acid long peptide (myrstic acid GGGDWLKAFYDKVAEKLKEAFPDWAKAAYDKAAEKAKEAA) referred to as acyl-5A was synthesized. This peptide is based on the 5A peptide (Sethi, Stonik et al. 2008), a bi-helical peptide that specifically promotes cholesterol efflux from cells by the ABCA1 transporter, which when pre-complexed with phospholipids reduces atherosclerosis in mice (Amar, D'Souza et al. 2010). The present invention demonstrates that hydrocarbon chain attachment at a single site of an apolipoprotein mimetic peptide preserves its ability to efflux cholesterol by the ABCA1 transporter and enables it to efflux cholesterol by other mechanisms. This modification also results in a peptide that favorably modifies the lipoprotein serum profile in the same anti-atherogenic manner as an apolipoprotein non-covalently associated with phospholipids.

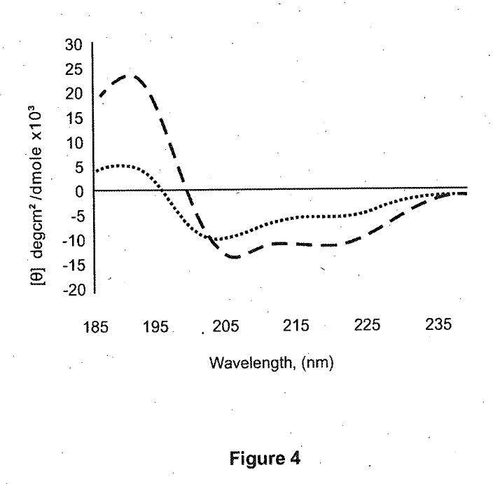

Acyl-5A has the same amino acid sequence of 5A except for the addition of a flexible three-chain glycine linker attached to the amino terminal end. In addition, it contains myristic acid, a 14-carbon fatty acid, attached via an amide bond to the amino terminal end of the glycine linker region. In aqueous solution, acyl-5A was found to form oligomer containing approximately 10 peptides per complex. By CD spectroscopy, acyl-5A at 24° C. was 36.7% helical, whereas lipid-free 5A was only 17.7% helical (FIG. 4).

### Example 5

### Assessment of Cholesterol Efflux by Stapled Peptides

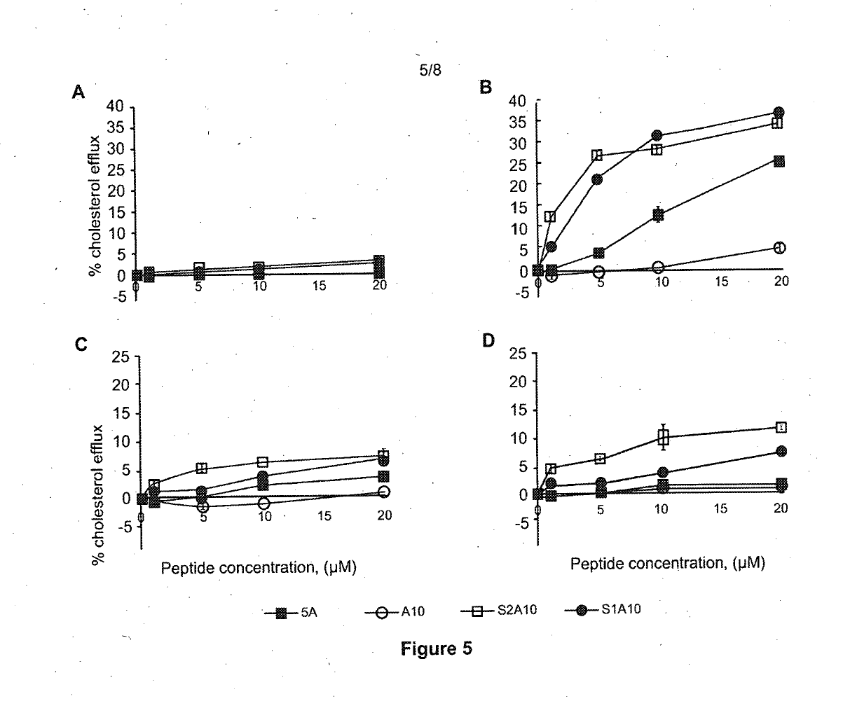

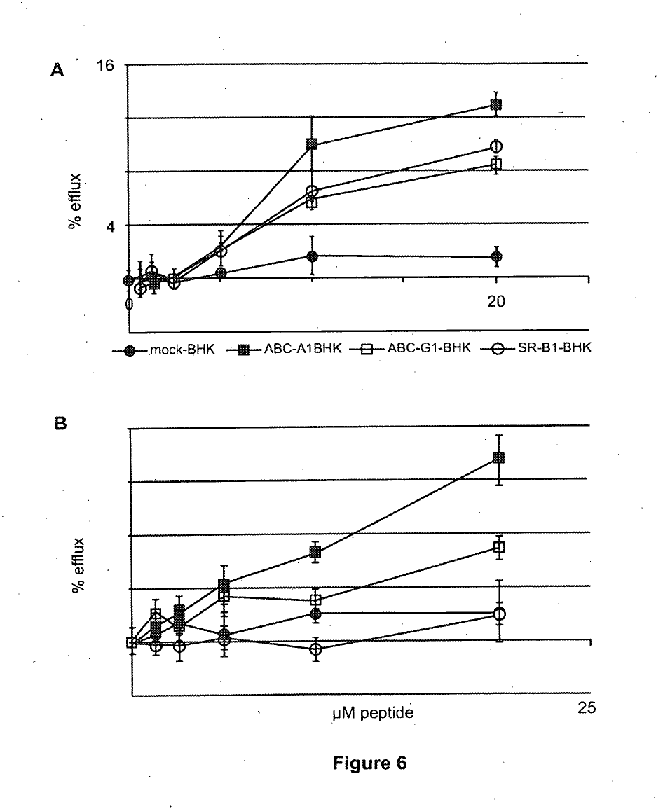

The ability of the three peptides and the bi-helical 5A peptide to promote cholesterol efflux was tested on BHK cells stably transfected with human ABCA1, ABCG1 or the human SRBI receptor (FIGS. 5 and 6). Cholesterol efflux studies were performed, as described previously. Control BHK cells and BHK cells stably transfected with either human ABCA1, ABCG1 or SR-BI were radiolabeled with [³H] cholesterol for 24 h, washed and then treated with the indicated concentration of peptides for 18 h. Percent efflux was calculated by subtracting the radioactive counts in blank medium (alpha-minimal essential media plus 10 μg/ml bovine serum albumin) from the radioactive counts in the presence of a peptide and then dividing the result by the sum of the radioactive counts in the medium plus the cell fraction.

For non-transfected BHK control cells, none of the peptides were able to promote significant amounts of cholesterol efflux (FIG. 5A), indicating that they cannot remove cholesterol by a nonspecific detergent-like process unlike some other more hydrophobic apolipoprotein mimetic peptides. A10 was ineffective in promoting cholesterol efflux by the ABCA1 transporter (FIG. 5A) or by any other mechanism (FIG. 5B-D). In contrast, after hydrocarbon stapling, S1A10 and S2A10 showed more than a 10-fold increase in cholesterol efflux (FIG. 5B). Furthermore, the stapled peptides showed greater cholesterol efflux, particularly at lower doses, than the much longer 5A bi-helical non-stapled peptide, which was previously designed for specifically effluxing cholesterol by the ABCA1 transporter and was shown to decrease atherosclerosis in mice.

Cholesterol efflux can also occur by other mechanisms, involving the ABCG1 transporter and the SR-BI receptor, as well as by passive exchange. Non-ABCA1 cholesterol efflux usually depends on the presence of a cholesterol acceptor that contains phospholipid into which cholesterol can be solubilized. Relative to A10 and 5A, the stapled peptides possess an ability to stimulate cholesterol efflux even without reconstitution with phospholipids (FIGS. 5C and D).

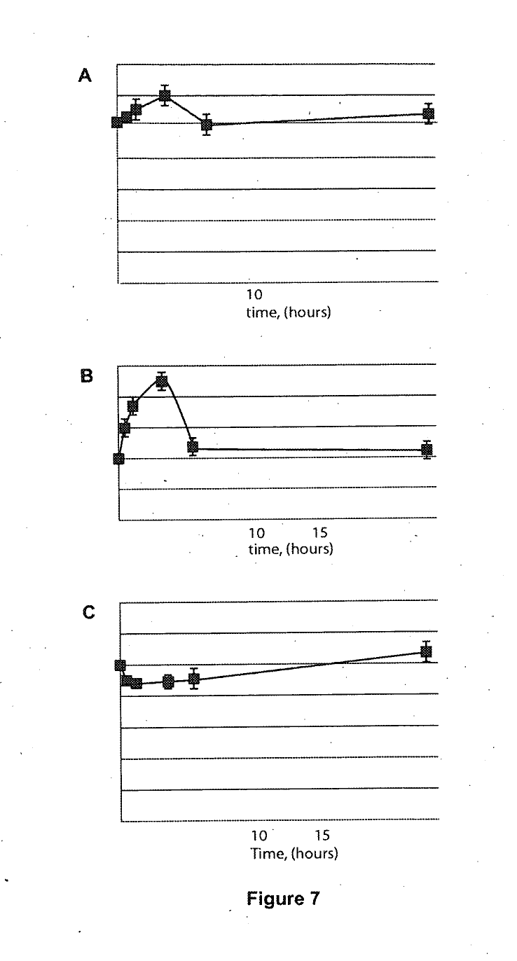

In FIG. 7, the results of testing the effect of acyl-5A on the serum lipid profile of mice are shown. When tested at a dose of 30 mg/kg, acyl-5A showed a very similar effect as was observed with lipid-free 5A at the same dose, which was shown to reduce atherosclerosis (Amar, D'Souza et al. 2010). It increased total cholesterol by approximately 20%, and there was a more than a 2-fold increase in free cholesterol, which was found primarily on HDL, thus demonstrating evidence for an increase in vivo cholesterol efflux (Amar, D'Souza et al. 2010).

Table 1. Examples of apolipoprotein mimetic peptides modified by hydrocarbon staples. Bolded/underlined residues indicate stapled linker region. The first and last residue in linker region would be substituted with a modified amino acid linker, containing a hydrocarbon bond, as described in FIG. 1, to form a covalent cross link between the first and last residue in the linker region. For cross links spanning 4 residues, they can be made with the following modified amino acids containing the hydrocarbon linkers: (S) alpha-methyl,alpha-pentenylglycine and (R) alphamethyl,alpha-pentenylglycine. For cross links spanning 5 residues, they can be made with the following modified amino acids containing the hydrocarbon linkers: (S)-a(4′-pentenyl)Ala and (S)-a-(4′-pentenyl)Ala. For cross links spanning 8 residues, they can be made with can be made with the following modified amino acids containing the hydrocarbon linkers: (S)-a-(4′-pentenyl)Ala and (R)-a-(Toctanyl)Ala. The modified amino acid linkers with the hydrocarbon chains can be cross linked by the olefin metathesis reaction with Bis(tricylcohexyl-phosphine)bezyldine ruthenium (IV) dichloride (Kim, Kutchukian et al. 2010). Peptides with more than one bolded interval, represent peptides containing more than one linker region. Names are designates so that first part indicates the sequence from a natural apolipoprotein. The next part indicates helix number from apolipoprotein. The third section containing numbers, correspond to the first and last amino position in the designated helix of the given apolipoprotein. Amino acid residues are listed from amino terminal to carboxy terminal end.

ApoA-I; Helix 1; 8-33
(SEQ ID NO: 1)
WDRVKDLATVYVDVLKDSGRDYVSQF
WDRVKDLATVYVDVLKDSGRDYVSQF
WDRVKDLATVYVDVLKDSGRDYVSQF
ApoA-I; Helix 2; 44-65
(SEQ. ID. NO: 2)
LKLLDNWDSVTSTFSKLREQL
LKLLDNWDSVTSTFSKLREQL
LKLLDNWDSVTSTFSKLREQL
LKLLDNWDSVTSTFSKLREQL
LKLLDNWDSVTSTFSKLREQL
ApoA-I; Helix 3; 66-87
(SEQ. ID. NO: 3)
PVTQEFWDNLEKETEGLRQEMS
PVTOEFWDNLEKETEGLRQEMS
PVTQEFWDNLEKETEGLRQEMS
PVTQEFWDNLEKETEGLRQEMS
ApoA-I; Helix 4: 88-98
(SEQ. ID. NO: 4)
KDLEEVKAKVQ
KDLEEVKAKVQ
ApoA-I; Helix 5; 99-120
(SEQ. ID. NO: 5)
PYLDDFQKKWQEEMELYRQKVE
PYLDDFQKKWQEEMELYROKVE
PYLDDFQKKWQEEMELYRQKVE
PYLDDFQKKWQEEMELYRQKVE
PYLDDFQKKWQEEMELYRQKVE
ApoA-I; Helix 5; 121-142
(SEQ. ID NO: 6)
PLRAELQEGARQKLHELQEKLS
PLRAELQEGARQKLHELQEKLS
PLRAELQEGARQKLHELQEKLS
PLRAELQEGARQKLHELQEKLS
PLRAELQEGARQKLHELQEKLS
PLRAELQEGAROKLHELQEKLS
ApoA-I; Helix 7; 143-164
(SEQ. ID. NO: 7)
PLGEEMRDRARAHVDALRTHLA
PLGEEMRDRARAHVDALRTHLA
PLGEEMRDRARAHVDALRTHLA
PLGEEMRDRARAHVDALRTHLA
PLGEEMRDRARAHVDALRTHLA
PLGEEMRDRARAHVDALRTHLA
PLGEEMRDRARAHVDALRTHLA
ApoA-I; Helix 8; 165-183
(SEQ. ID. NO: 8)
PYSDELRQRLAARLEALKENGG
PYSDELRORLAARLEALKENGG
PYSDELRQRLAARLEALKENGG
PYSDELRQRLAARLEALKENGG
PYSDELRQRLAARLEALKENGG
PYSDELRQRLAARLEALKENGG
PYSDELRQRLAARLEALKENGG
ApoA-I; Helix 9; 187-208
(SEQ. ID. NO: 9)
ARLAEYHAKATEHLSTLSEKAK
ARLAEYHAKATEHLSTLSEKAK
ARLAEYHAKATEHLSTLSEKAK
ARLAEYHAKATEHLSTLSEKAK
ARLAEYHAKATEHLSTLSEKAK
ApoA-I; Helix 10; 209-219
(SEQ. ID. NO: 10)
PALEDLRQGLL
PALEDLRQGLL
PALEDLRQGLL
ApoA-I; Helix 11; 220-243
(SEQ. ID. NO: 11)
PVLESFKVSFLSALEEYTKKLN
PVLESFKVSFLSALEEYTKKLN
PVLESFKVSFLSALEEYTKKLN
PVLESFKVSFLSALEEYTKKLN
PVLESFKVSFLSALEEYTKKLN
PVLESFKVSFLSALEEYTKKLN
PVLESFKVSFLSALEEYTKKLN
PVLESFKVSFLSALEEYTKKLN
PVLESFKVSFLSALEEYTKKLN
ApoA-II; Helix I; 7-30
(SEQ. ID. NO: 12)
TVLLLTICSLEGALVRRQAKEPCV
TVLLLTICSLEGALVRRQAKEPCV
TVLLLTICSLEGALVRRQAKEPCV
TVLLLTICSLEGALVRRQAKEPCV
TVLLLTICSLEGALVRRQAKEPCV
TVLLLTICSLEGALVRRQAKEPCV
ApoA-II; Helix 2; 39-50
(SEQ. ID. NO: 13)
QTVTDYGKDLME
ApoA-II; Helix 3; 51-71
(SEQ. ID. NO: 14)
KVKSPELQAEAKSYFEKSKE
ApoA-IV; Helix 1; 7-31
(SEQ. ID. NO: 15)
VLTLALVAVAGARAEVSADQVATV
VLTLALVAVAGARAEVSADOVATV
VLTLALVAVAGARAEVSADQVATV
VLTLALVAVAGARAEVSADQVATV
VLTLALVAVAGARAEVSADQVATV
VLTLALVAVAGARAEVSADQVATV
VLTLALVAVAGARAEVSADQVATV
VLTLALVAVAGARAEVSADQVATV
ApoA-IV; Helix 2; 40-61
(SEQ. ID. NO: 16)
NNAKEAVEHLQKSELTQQLNAL
NNAKEAVEHLQKSELTQQLNAL
NNAKEAVEHLQKSELTQQLNAL
NNAKEAVEHLQKSELTQQLNAL
NNAKEAVEHLQKSELTQQLNAL
NNAKEAVEHLQKSELTQQLNAL
ApoC-I; Helix 1:7-32
(SEQ. ID. NO: 17)
LPVLVVVLSIVLEGPAPAQGTPDVSS
LPVLVVVLSIVLEGPAPAQGTPDVSS
ApoC-I; Helix 2; 33-53
(SEQ. ID. NO: 18)
ALDKLKEFGNTLEDKARELIS
ALDKLKEFGNTLEDKARELIS
ALDKLKEFGNTLEDKARELIS
ApoC-III; Helix 2; 28-49
(SEQ. ID. NO: 19)
VVALLALLASARASEAEDASLL
VVALLALLASARASEAEDASLL
VVALLALLASARASEAEDASLL
VVALLALLASARASEAEDASLL
VVALLALLASARASEAEDASLL
VVALLALLASARASEAEDASLL
VVALLALLASARASEAEDASLL
ApoE; Helix 2; 158-182
(SEQ. ID. NO: 20)
HLRKLRKRLLRDADDLQKRLAVYQA
HLRKLRKRLLRDADDLQKRLAVYQA
HLRKLRKRLLRDADDLQKRLAVYQA
HLRKLRKRLLRDADDLQKRLAVYQA
HLRKLRKRLLRDADDLQKRLAVYQA
ApoE; Helix 4; 26-48
(SEQ. ID. NO: 21)
AQAWGERLRARMEEMGSRTRDR
ApoE; Helix 5; 249-266
(SEQ. ID. NO: 22)
LDEVKEQVAEVRAKLEEQAQ
LDEVKEQVAEVRAKLEEQAQ
LDEVKEQVAEVRAKLEEQAQ
18A synthetic consensus peptide;
Helix 1; 1-18
(SEQ. ID. NO: 23)
DWLKAFYDKVAEKLKEAF
DWLKAFYDKVAEKLKEAF
DWLKAFYDKVAEKLKEAF
DWLKAFYDKVAEKLKEAF
DWLKAFYDKVAEKLKEAF
DWLKAFYDKVAEKLKEAF
DWLKAFYDKVAEKLKEAF

### REFERENCES

All references are incorporated by reference in their entirety.
Amar, M. J., 1W. D'Souza, et al. (2010). “SA apolipoprotein mimetic peptide promotes cholesterol efflux and reduces atherosclerosis in mice J Pharmacol Exp Ther 334(2):634-41.
Bhattacharya, S., H. Zhang, et al. (2008). “Solution structure of a hydrocarbon stapled peptide inhibitor in complex with monomeric C-terminal domain of HIV-1 capsid.” J BioI Chern 283(24): 16274-16278.
Bielicki, J. K., H. Zhang, et al. (2010). “A new HDL mimetic peptide that stimulates cellular cholesterol efflux with high efficiency greatly reduces atherosclerosis in mice.” J Lipid Res 51(6): 1496-1503.
Bloedon, L. T., R. Dunbar, et al. (2008). “Safety, pharmacokinetics, and pharmacodynamics of oral apoA-I mimetic peptide D-4F in high-risk cardiovascular patients.” I Lipid Res 49(6): 1344-1352.
Brewer, H. B., Jr., A. T. Remaley, et al. (2004). “Regulation of plasma high density lipoprotein levels by the ABCA1 transporter and the emerging role of high-density lipoprotein in the treatment of cardiovascular disease.” Arterioscler Thromb Vasc Biol 24(10): 1755-1760.
Buga, G. M., J. S. Frank, et al. (2006). “D-4F decreases brain arteriole inflammation and improves cognitive performance in LDL receptor-null mice on a Western diet.” J Lipid Res 47(10): 2148-2160.
D'Souza, W., J. A. Stonik, et al. (2010). “Structure/function relationships of apolipoprotein a-1 mimetic peptides: implications for antiatherogenic activities of high-density lipoprotein.” Circ Res 107(2): 217-227.
Frank, P. G. and Y. L. Marcel (2000). “Apolipoprotein A-I: structure-function relationships.” Lipid Res 41(6): 853-872.
Henchey, L. K., A. L. Jochim, et al. (2008). “Contemporary strategies for the stabilization of peptides in the alpha-helical conformation.” Curr Opin Chem Bioi 12(6): 692-697.
Khera, A. V., M. Cuchel, et al. (2011). “Cholesterol efflux capacity, high-density lipoprotein function, and atherosclerosis.” N Engl J Med 364(2): 127-135.
Kim, Y. W., P. W. Kutchukian, G. L. Verdine (2010). “Introduction of a hydrocarbon, i,i+3 staples into alpha-helices via ring-closing olefin metathesis. Org Lett 12(13): 3046-9.
Klon, A. E., J. P. Segrest, et al. (2002). “Comparative models for human apolipoprotein A-I bound to lipid in discoidal high-density lipoprotein particles.” Biochemistry 41(36): 10895-10905.
Kutchukian, P. S., J. S. Yang, et al. (2009). “All-Atom Model for Stabilization of alpha-Helical Structure in Peptides by Hydrocarbon Staples.” Journal of the American Chemical Society 131(13): 4622-4627.
Lund-Katz, S, and M. C. Phillips (2010) “High density lipoprotein structure function and role in reverse cholesterol transport.” Sub cell Biochem 51: 183-227.
Mendez, A. J., G. M. Anantharamaiah, et al. (1994). “Synthetic amphipathic helical peptides that mimic apolipoprotein A-I in clearing cellular cholesterol.” J Clin Invest 94(4): 1698-170.5.
Navab, M., S. T. Reddy, et al. (2011). “HDL and cardiovascular disease: atherogenic and atheroprotective mechanisms.” Nat Rev Cardiol.
Navab, M., P. Ruchala, et al. (2009) “A novel method for oral delivery of apolipoprotein mimetic peptides synthesized from all L-amino acids.” Lipid Res 50(8): 1538-1547.
Nestor, Y. J. (2009) “The medicinal chemistry of peptides.” Curr Med Chem 16(33):4399-418.
Nissen, S. E. (2005) “Effect of intensive lipid lowering on progression of coronary atherosclerosis: evidence for an early benefit from the Reversal of Atherosclerosis with Aggressive Lipid Lowering (REVERSAL) trial.” Am J. Cardio 96(5A): 61F-68F.
Osei-Hwedieh, D. O., M. Amar, et al. (2011) “Apolipoprotein mimetic peptides: Mechanisms of action as anti-atherogenic agents.” Pharmacol Ther 130(1):83-91.
Panagotopulos, S. E., S. R. Witting, et al. (2002) “The role of apolipoprotein A-I helix 10. in apolipoprotein-mediated cholesterol efflux via the ATP binding cassette transporter ABCA1.” Biol Chem 277(42): 3947739484.
Phillips, M. C., K. L. Gillotte, et al. (1998). “Mechanisms of high density lipoprotein-mediated efflux of cholesterol from cell plasma membranes.” Atherosclerosis 137 Suppl: S13-17.
Remaley, A. T., M. Amar, et al. (2008) “HDL-replacement therapy: mechanism of action, types of agents and potential clinical indications.” Expert Rev Cardiovasc Ther 6(9): 120.3-1215.
Remaley, A. T., F. Thomas, et al. (2003). “Synthetic amphipathic helical peptides promote lipid efflux from cells by an ABCA1˜dependent and an ABCA1˜independent pathway.” J Lipid Res 44(4): 828-836.
Remaley A T, W. G. (2008). “High˜density lipoprotein: what is the best way to measure its antiathrerogenic potential.” Expert Opinion on Medical Diagnostics 2(7): 773-788.
Rothblat, G. H. and M. C. Phillips (2010). “High-density lipoprotein heterogeneity and function in reverse cholesterol transport.” Curr Opin Lipid 21(3): 229˜238.
Sethi, A. A., M. Amar, et al. (2007). “Apolipoprotein AI mimetic peptides: possible new agents for the treatment of atherosclerosis.” Curr Opin Investig Drugs 8(3): 201-212.
Sethi, A. A., J. A. Stonik (2008). “Asymmetry in the lipid affinity of bihelical amphipathic peptides. A structural determinant for the specificity of ABCA1-dependent cholesterol efflux by peptides.” J Biol Chem 283(47):32273-S2.
Shaw, J. A., A. Bobik, et al. (2008). “Infusion of reconstituted high-density lipoprotein leads to acute changes in human atherosclerotic plaque.” Circ Res 103(10): 1084-1091.
Smith, J. c., H. J. Pownall, et al. (1978). “The plasma lipoproteins: structure and metabolism.” Annu Rev Biochem 47: 751˜757.
Tardif, J. c., J. Gregoire, et al. (2007). “Effects of reconstituted high-density lipoprotein infusions on coronary atherosclerosis: a randomized controlled trial.” JAMA 297(15): 1675-1682.
Watson, C. E., N. Weissbach, et al. (2011). “Treatment of patients with cardiovascular disease with L-4F, an apo-A1 mimetic, did not improve select biomarkers of HDL function.” J Lipid Res 52(2): 361-373.
Wool, G. D., C. A. Reardon, et al. (2008). “Apolipoprotein A-I mimetic peptide helix number and helix linker influence potentially anti-atherogenic properties.” I Lipid Res 49(6): 1268-1283.

We claim:

1. A stapled apolipoprotein mimetic peptide, wherein the peptide comprises S1A10 or S2A10.

2. A pharmaceutical formulation comprising the stapled apolipoprotein mimetic peptide of claim 1.

3. A method of treating a cardiovascular disease or inflammation in a subject comprising administering a therapeutically effective amount of the pharmaceutical formulation of claim 2.

4. The method of claim 3, wherein the subject is a mammal.

5. The method of claim 4, wherein the subject is a human.

6. The method of claim 3, wherein the administration is oral, parenteral, by intramuscular injection, by intraperitoneal injection, or transdermal.

7. The method of claim 6, wherein the administration is oral.

8. A composition comprising a hydrocarbon stapled apolipoprotein mimetic peptide.

9. The composition of claim 8, wherein the hydrocarbon stapled apolipoprotein mimetic peptide is substantially pure.


SEQUENCE LISTING (25 sequences)

SEQ ID NO: 1 - protein, 78 aa, Homo sapiens
WDRVKDLATVYVDVLKDSGRDYVSQFWDRVKDLATVYVDVLKDSGRDYVSQFWDRVKDLATVYVDVLKDSGRDYVSQF

SEQ ID NO: 2 - protein, 105 aa, Homo sapiens
LKLLDNWDSVTSTFSKLREQLLKLLDNWDSVTSTFSKLREQLLKLLDNWDSVTSTFSKLREQLLKLLDNWDSVTSTFSKLREQLLKLLDNWDSVTSTFSKLREQL

SEQ ID NO: 3 - protein, 88 aa, Homo sapiens
PVTQEFWDNLEKETEGLRQEMSPVTQEFWDNLEKETEGLRQEMSPVTQEFWDNLEKETEGLRQEMSPVTQEFWDNLEKETEGLRQEMS

SEQ ID NO: 4 - protein, 22 aa, Homo sapiens
KDLEEVKAKVQKDLEEVKAKVQ

SEQ ID NO: 5 - protein, 110 aa, Homo sapiens
PYLDDFQKKWQEEMELYRQKVEPYLDDFQKKWQEEMELYRQKVEPYLDDFQKKWQEEMELYRQKVEPYLDDFQKKWQEEMELYRQKVEPYLDDFQKKWQEEMELYRQKVE

SEQ ID NO: 6 - protein, 132 aa, Homo sapiens
PLRAELQEGARQKLHELQEKLSPLRAELQEGARQKLHELQEKLSPLRAELQEGARQKLHELQEKLSPLRAELQEGARQKLHELQEKLSPLRAELQEGARQKLHELQEKLSPLRAELQEGARQKLHELQEKLS

SEQ ID NO: 7 - protein, 154 aa, Homo sapiens
PLGEEMRDRARAHVDALRTHLAPLGEEMRDRARAHVDALRTHLAPLGEEMRDRARAHVDALRTHLAPLGEEMRDRARAHVDALRTHLAPLGEEMRDRARAHVDALRTHLAPLGEEMRDRARAHVDALRTHLAPLGEEMRDRARAHVDALRTHLA

SEQ ID NO: 8 - protein, 154 aa, Homo sapiens
PYSDELRQRLAARLEALKENGGPYSDELRQRLAARLEALKENGGPYSDELRQRLAARLEALKENGGPYSDELRQRLAARLEALKENGGPYSDELRQRLAARLEALKENGGPYSDELRQRLAARLEALKENGGPYSDELRQRLAARLEALKENGG

SEQ ID NO: 9 - protein, 110 aa, Homo sapiens
ARLAEYHAKATEHLSTLSEKAKARLAEYHAKATEHLSTLSEKAKARLAEYHAKATEHLSTLSEKAKARLAEYHAKATEHLSTLSEKAKARLAEYHAKATEHLSTLSEKAK

SEQ ID NO: 10 - protein, 33 aa, Homo sapiens
PALEDLRQGLLPALEDLRQGLLPALEDLRQGLL

SEQ ID NO: 11 - protein, 198 aa, Homo sapiens
PVLESFKVSFLSALEEYTKKLNPVLESFKVSFLSALEEYTKKLNPVLESFKVSFLSALEEYTKKLNPVLESFKVSFLSALEEYTKKLNPVLESFKVSFLSALEEYTKKLNPVLESFKVSFLSALEEYTKKLNPVLESFKVSFLSALEEYTKKLNPVLESFKVSFLSALEEYTKKLNPVLESFKVSFLSALEEYTKKLN

SEQ ID NO: 12 - protein, 144 aa, Homo sapiens
TVLLLTICSLEGALVRRQAKEPCVTVLLLTICSLEGALVRRQAKEPCVTVLLLTICSLEGALVRRQAKEPCVTVLLLTICSLEGALVRRQAKEPCVTVLLLTICSLEGALVRRQAKEPCVTVLLLTICSLEGALVRRQAKEPCV

SEQ ID NO: 13 - protein, 12 aa, Homo sapiens
QTVTDYGKDLME

SEQ ID NO: 14 - protein, 20 aa, Homo sapiens
KVKSPELQAEAKSYFEKSKE

SEQ ID NO: 15 - protein, 192 aa, Homo sapiens
VLTLALVAVAGARAEVSADQVATVVLTLALVAVAGARAEVSADQVATVVLTLALVAVAGARAEVSADQVATVVLTLALVAVAGARAEVSADQVATVVLTLALVAVAGARAEVSADQVATVVLTLALVAVAGARAEVSADQVATVVLTLALVAVAGARAEVSADQVATVVLTLALVAVAGARAEVSADQVATV

SEQ ID NO: 16 - protein, 132 aa, Homo sapiens
NNAKEAVEHLQKSELTQQLNALNNAKEAVEHLQKSELTQQLNALNNAKEAVEHLQKSELTQQLNALNNAKEAVEHLQKSELTQQLNALNNAKEAVEHLQKSELTQQLNALNNAKEAVEHLQKSELTQQLNAL

SEQ ID NO: 17 - protein, 52 aa, Homo sapiens
LPVLVVVLSIVLEGPAPAQGTPDVSSLPVLVVVLSIVLEGPAPAQGTPDVSS

SEQ ID NO: 18 - protein, 63 aa, Homo sapiens
ALDKLKEFGNTLEDKARELISALDKLKEFGNTLEDKARELISALDKLKEFGNTLEDKARELIS

SEQ ID NO: 19 - protein, 154 aa, Homo sapiens
VVALLALLASARASEAEDASLLVVALLALLASARASEAEDASLLVVALLALLASARASEAEDASLLVVALLALLASARASEAEDASLLVVALLALLASARASEAEDASLLVVALLALLASARASEAEDASLLVVALLALLASARASEAEDASLL

SEQ ID NO: 20 - protein, 125 aa, Homo sapiens
HLRKLRKRLLRDADDLQKRLAVYQAHLRKLRKRLLRDADDLQKRLAVYQAHLRKLRKRLLRDADDLQKRLAVYQAHLRKLRKRLLRDADDLQKRLAVYQAHLRKLRKRLLRDADDLQKRLAVYQA

SEQ ID NO: 21 - protein, 22 aa, Homo sapiens
AQAWGERLRARMEEMGSRTRDR

SEQ ID NO: 22 - protein, 60 aa, Homo sapiens
LDEVKEQVAEVRAKLEEQAQLDEVKEQVAEVRAKLEEQAQLDEVKEQVAEVRAKLEEQAQ

SEQ ID NO: 23 - protein, 126 aa, Artificial Sequence
  18A synthetic consensus peptide; Helix 1; 1-18
DWLKAFYDKVAEKLKEAFDWLKAFYDKVAEKLKEAFDWLKAFYDKVAEKLKEAFDWLKAFYDKVAEKLKEAFDWLKAFYDKVAEKLKEAFDWLKAFYDKVAEKLKEAFDWLKAFYDKVAEKLKEAF

SEQ ID NO: 24 - protein, 40 aa, Artificial Sequence
  bi-helical peptide that specifically promotes cholesterol efflux from cells by the ABCA1 transporter
GGGDWLKAFYDKVAEKLKEAFPDWAKAAYDKAAEKAKEAA

SEQ ID NO: 25 - protein, 23 aa, Homo sapiens
  MOD_RES (1)..(1): AMIDATION
VLESFKVSFLSALEEYTKKLNTQ